(19) United States
(12) Patent Application Publication
(10) Pub. No.: US 2010/0198236 A1
(43) Pub. Date: Aug. 5, 2010

(54) Surgical Meshes and Methods of Use

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/698,188

(22) Filed: Feb. 2, 2010

Related U.S. Application Data
(60) Provisional application No. 61/149,080, filed on Feb. 2, 2009.

(51) Int. Cl. A61B 17/00

(52) U.S. Cl. 606/151

(57) ABSTRACT

Surgical meshes having a plurality of aperture reducing a patient's foreign-body reaction and thereby reducing foreign body inoculum at a surgical site. The plurality of apertures within the surgical meshes may be placed against or immediately adjacent a surgical site or incision. The plurality of apertures may be placed within the mesh to avoid critical knots or loops and thus the creation of the plurality of apertures will not weaken or unravel the affected portions of the mesh. Placement of such apertures in close proximity to the surgical site allows for a greater reduction in erosion, extrusion, and incision dehiscence. The surgical meshes of the present invention may comprise a hybrid of materials including at least one permanent material and at least one absorbable material. The scope of the present invention includes the disclosed surgical meshes, their beneficial composition and method of manufacture, and their methods of use.

### CROSS REFERENCE TO RELATED APPLICATIONS

This application claims the benefit of provisional application Ser. No. 61/149,080, filed with the U.S. Patent and Trademark Office on Feb. 2, 2009, which is hereby incorporated by references in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention relates generally to implantable surgical meshes and their methods of use, and more particularly, to implantable surgical meshes that may contain a plurality of apertures disposed either directly against or immediately adjacent a surgical site thereby reducing inoculums and/or other foreign bodies.

2. Background Art

Hernia repairs are among the more common surgical operations that may employ a mesh fabric prosthesis. Such mesh fabric prostheses may also be used in other surgical procedures including the repair of anatomical defects of the abdominal wall, diaphragm, and chest wall, correction of defects in the genitourinary system, and repair of traumatically damaged organs such as the spleen, liver or kidney.

Surgically implantable mesh patches for the repair of inguinal and other abdominal wall hernias, which are intended for permanent placement within a patient's body space, have been provided and used previously. Tension free surgical repairs of hernias have been developed using synthetic mesh materials to bridge and to patch hernia defects. These repairs resulted in both a decrease in the recurrence rate as well as a decrease in the amount of a patient's post operative discomfort. Patients undergoing these more advanced procedures were able and are able to resume their normal activities sooner.

Some of these earlier techniques are somewhat complicated. Several use a plug or a locating member to fit within the hernia defect itself. Also many of these earlier techniques were designed specifically for use in laparoscopic repair of hernias. Moreover, many of the prior inventions required suturing to the patient's body tissue. Although these medical advances are acknowledged for their usefulness and success, there remains a need or needs for more improvements in the surgical repair of hernias.

In surgical repairs such as hernia repair, pelvic floor repair, urethral slings for treating incontinence, and many others, a woven or knitted mesh structure may be desirable in that it allows tissue ingrowth into and through the mesh. The tissue ingrowth may be in the form of a tissue fibrosis, where non-oriented tissue cells invade the mesh and grow in a random, disorganized fashion. The combination of mesh and ingrown tissue, however, may produce a relatively hard, inflexible construction that does not resemble the tissue structure that it is reinforcing or replacing. This may be due, in part, to the fact that the mesh structure in combination with the random ingrowth pattern of the tissue does not reflect the natural, organized cell structure in the absence of the implanted foreign body or mesh. Thus, the resulting relatively inflexible structure may lead to tissue erosion problems in proximity to the implant or to organs in the vicinity of the implant.

Surgical meshes commonly incorporate synthetic and natural materials to assist in surgically repairing anatomic defects. These materials are typically delivered as sheet-like structures. These sheets can cure the hernia via two mechanisms. The sheets, when permanent, provide a sustained covering of the anatomic defect. In addition, sheets are porous and therefore serve as templates for ingrowth of the patient's tissue creating a natural repair. Although the practice of covering hernias with sheets of natural or synthetic material has lead to a substantial improvement in the surgical treatment of hernias including shorter surgical times, less pain, less deformity, and lower failure rates, erosion of the material has continued to be a substantial problem.

The implanted material, often a synthetic mesh, may become exposed after surgery. This typically occurs secondary to the separation of the skin or mucosal incision. Various terms have been used to describe this phenomenon including erosion, extrusion, and incision dehiscence. Although there are several possible causes for extrusion, it most likely occurs secondary to a foreign-body reaction, the body reacting against a foreign substance. This inflammatory response leads to extrusion. It has been hypothesized that this inflammatory response is proportional to the inoculum of implanted material.

Recent attempts to decrease the inoculum have focused on decreasing the material density. This has been accomplished by using thinner or fewer numbers of fibers when weaving or braiding the surgical mesh. Also, changing the number or type of knots or loops used to create the material may further serve to decrease the density of the material. These manufacturing methods, which typically create a uniform decrease in density, have not lead to a substantial decrease in the incidence of extrusion. Prior art attempts can not decrease the density of the foreign body or surgical mesh below a critical level responsible for a strong foreign-body type reaction. Attempts to further decrease the density with presently used methods have lead to weaknesses in the surgical material that are capable of compromising the surgical repair itself. A need exists for an improved surgical mesh, a manner of manufacturing such improved surgical meshes, and a method of utilizing these innovative meshes to the benefit of all patients requiring surgical repair.

### BRIEF DESCRIPTION OF THE DRAWINGS

A better understanding of the present invention will be realized from the detailed description that follows, taken in conjunction with the accompanying drawings, in which:

FIG. 1 depicts a top view of one embodiment of a surgical mesh of the present invention comprising a plurality of apertures.

FIG. 2 depicts a top view of another embodiment of a surgical mesh of the present invention comprising a plurality of apertures.

FIG. 3 depicts a side view of one embodiment of a surgical mesh of the present invention being disposed adjacent an incision within a patient.

FIG. 4 depicts a planar view of one embodiment of an initial step of a method of the present invention.

FIG. 5 depicts a planar view of one embodiment of another step of the method of the present invention.

FIG. 6 depicts a planar view of an embodiment of still another step of the method of the present invention.

### DETAILED DESCRIPTION OF THE INVENTION

Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

The present invention provides for surgical meshes and methods providing for a decrease in the inoculum of suspect foreign material below the critical level needed to prevent implant extrusion without compromising the strength and integrity of the surgical repair. Surgical procedures that may benefit from the devices and method of the present invention include but are not limited to hernia repairs, pelvic floor repairs, urethral slings for treating incontinence, other tissue weakness repairs, and the like.

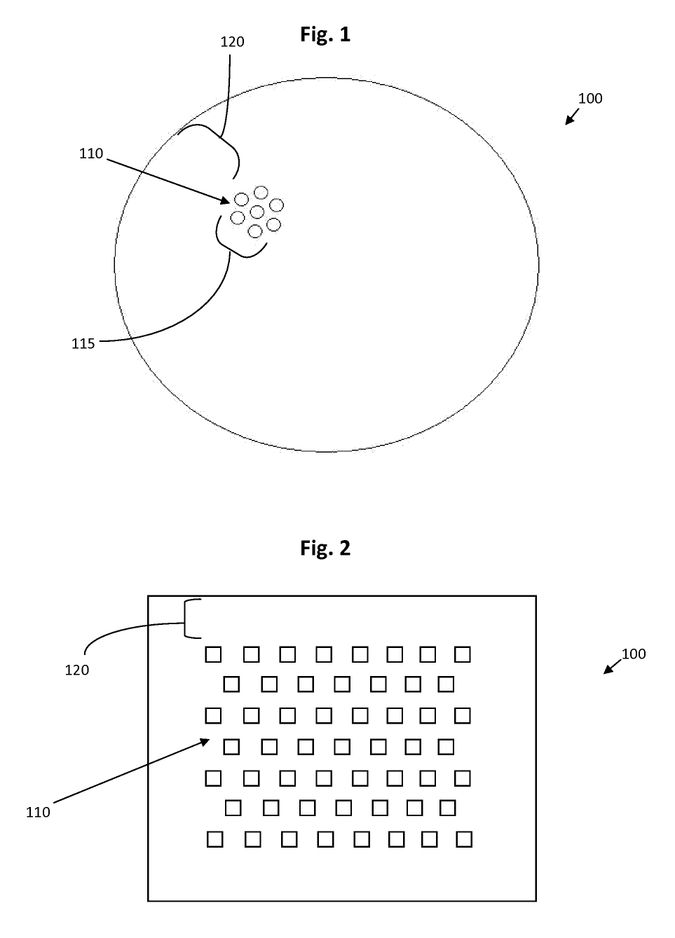

Prior art attempts at decreasing extrusion have focused on lowering the material density of the implanted material and providing one or more holes having no specific orientation to allow for generalized tissue ingrowth. To remedy the industry need in a different manner, the present invention utilizes both a novel configuration and method of use to reduce a patient's foreign-body reaction and thereby foreign body inoculum at a specific location. As depicted in FIG. 1, the present invention comprises a surgical mesh 100 having a plurality of apertures 110 for reducing foreign body inoculum about a surgical site. The location of the plurality of apertures 110 upon the surgical mesh 100 may be variable and may depend on the type and goals of each respective surgical procedure as well as the anatomy of the particular patient. The plurality of apertures 110 may be clustered on the surgical mesh 100 to form a treatment zone 115 that may serve to focus their functionality on a specific surgical site when the surgical mesh 100 is placed on or within the patient. In a preferred embodiment, the peripheral edges, circumference, or outer border 120 of the surgical mesh 100 may be void of apertures 110 so as not to structurally weaken the areas of the surgical mesh 100 that may be fixed to a patient via sutures, tacks, or the like.

As depicted in FIGS. 1-2, the overall the overall shape and configuration of both the surgical mesh 100 and plurality of apertures 110 is not a limitation of the present invention. Any manner of configuration may be used provided that the plurality of apertures 110 are disposed through the surgical mesh 100 so that the plurality of apertures 110 may be disposed on or adjacent a surgical site or incision. Given the many variables involved in any given surgical procedure, the shape of the surgical mesh 100 and shape of the cluster of the plurality of apertures 110 may be adjusted and optimized for the specific circumstances of each respective surgical procedure. While the primary focus of the present invention is to position the plurality of apertures 110 directly adjacent to the surgical site or incision, the scope of the invention may further include one or more secondary apertures spread throughout the mesh 100 to decrease the foreign body inoculum away from the surgical site or incision as well.

The surgical mesh 100 of the present invention may comprise any materials known within the art and be manufactured in any manner known within the art, such as with a conventional braiding process. Holes for ingrowth through surgical meshes of the prior art are disclosed to have been created by mechanical means such as a simple hole punch. Such mechanical damage and cutting applied to the prior art surgical mesh may serve to unravel the surgical mesh while further exposing patient tissue to a greater number of sharp ends of the cut mesh material. The holes found within the devices of the prior art serve to provide potential locations of tissue ingrowth and are disclosed to be positioned about the periphery of the surgical mesh thereby providing a point through which instruments or clips may be placed.

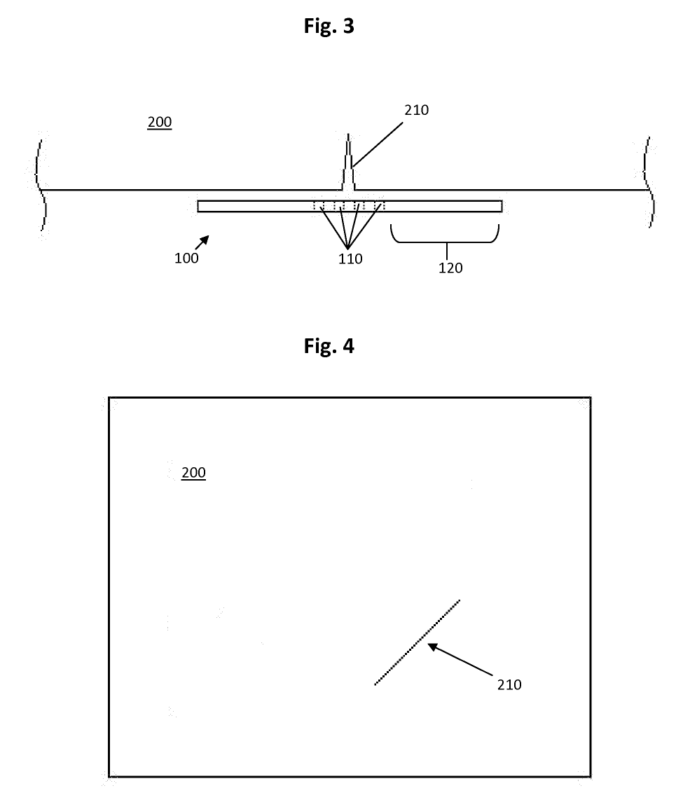

In one embodiment of the present invention, the surgical mesh 100 may be provided with a plurality of apertures 110 formed in a manner that will not reduce or compromise the structural integrity of the surgical mesh 100 while such apertures 110 may beneficially decrease the inoculum near an incision serving to decrease both peri-incisional inflammation and incision separation. As illustrated in FIGS. 1-2, the plurality of apertures 110 may be formed by creating holes of uniform or varying geometries or sizes at one or more regions about the surgical mesh 100. In a preferred embodiment, the holes or apertures 110 may be concentrated in the area of the surgical mesh 100 that will lay against the surgical site or incision 210 (see FIG. 3). The size, geometry, and spacing of such holes or apertures 110 may be limited by the material properties of the mesh or other surgical material within which they are formed. In a preferred embodiment, the formation of the plurality of holes or apertures 110 may typically be performed after the braiding, weaving of the mesh 100, or the manufacturing or production of other sheet material.

To provide a reduced material density within the mesh 100 near an incision 210, the plurality of apertures 110 may be formed via a number of possible techniques. The prior art act of utilizing a simple hole punch to generate random tissue ingrowth holes acts to both weaken the strength of the surgical mesh and create additional sharp ends at the incision site. In the meshes and methods of the present invention, the plurality of apertures 110 within the surgical mesh 100 may be formed by any manner known within the art including but not limited to the application of laser, ultrasound, hot dye, or any other energy source known within the art. Use of such an energy source as a means with which to create the plurality of apertures 110 may serve to reduce the surgical mesh material density only near the incision 210 and/or surgical site. Additionally, apertures 110 may also be placed in other areas of the mesh 100. The energy source may further act to seal or melt the loose “sharp” ends of the surgical mesh 100 exposed about the newly formed apertures 110, thereby retaining the structure and integrity of the surgical mesh 100 and potentially rounding or smoothing any sharp ends. Given the deliberate placement of the apertures 110, the apertures 110 may be strategically placed to avoid critical knots or loops that, if cut, would unwind the surgical mesh 100 material. The remaining mesh 100 material will be of substantial strength and will not be prone to tearing or pull out.

In a second embodiment of the present invention, the density of the surgical mesh 100 may be decreased due to the material composition of the surgical mesh 100 implanted during the procedure. In such a second embodiment, the surgical mesh 100 of the present invention may comprise at least one permanent material and at least one absorbable material. In this manner, the inoculum of any suspect foreign material near an incision may be reduced below the critical level responsible for extrusion by replacing some of the mesh 100 material with a less reactive and less foreign material. The number and/or size of the respective fibers of the suspect material may then successfully be reduced to a density that would normally compromise the strength of the material and, hence, the strength and integrity of a hernia repair performed in accordance with the prior art.

The braiding, weaving, or other manufacturing process used to create the second embodiment of the present invention may involve the creation of a hybrid material. One or more less reactive materials may be combined with the suspect material. Conventionally, polypropylene is the most commonly used fiber in surgical meshes used to treat hernias. Using polypropylene as an example, the number and/or size of polypropylene fibers used in the creation of a sheet of surgical mesh may be decreased and another type of fiber or fibers may be introduced in order to maintain the strength of the mesh 100, sheet, or other material. Other less reactive fibers are typically less durable than the presently used suspect foreign fibers such as polypropylene. Hence, they have not been utilized as primary materials for hernia repair.

In the meshes 100 and methods of the present invention, the less reactive, less durable, fibers may be used in order to drop the inoculum of the suspect foreign material below the critical level responsible for extrusion. These less reactive, less durable fibers may comprise absorbable fibers and maintain the overall strength of the material for a sufficient time to allow tissue ingrowth. The resulting surgical mesh 100 comprised of hybrid materials will hence be associated with a minimal foreign-body type reaction, have sufficient short term strength to allow sufficient time for tissue ingrowth, and have a necessary amount of the more durable but more reactive fibers to reinforce or strengthen the hernia repair to an extent not possible by tissue ingrowth alone. The less reactive absorbable fibers may include fibers that comprise various blends and/or copolymers of polyglycolic acid (PGA), lactic acid, or caprolactone. Specific examples include but are not limited to the products sold by Ethicon, Inc. under the trade names MONOCRYL, VICRYL, and PDS and the products sold by Covidien AG under the trade name DEXON. In a preferred embodiment, the more reactive and permanent material (e.g. polypropylene) may comprise no less than ten percent (10%) and no more than ninety-five percent (95%) of the total surgical mesh 100 material.

In a third embodiment of the present invention, the plurality of apertures 110 within the surgical mesh 100 may be created by the addition of an accessory braid or weave pattern. The structure of the braid of weave pattern of the fibers comprising the surgical mesh 100 may act to form the plurality of apertures 110 without the need for creating the apertures 110 via the application of an energy source or the incorporation of an absorbable material within the fibers of the surgical mesh 100.

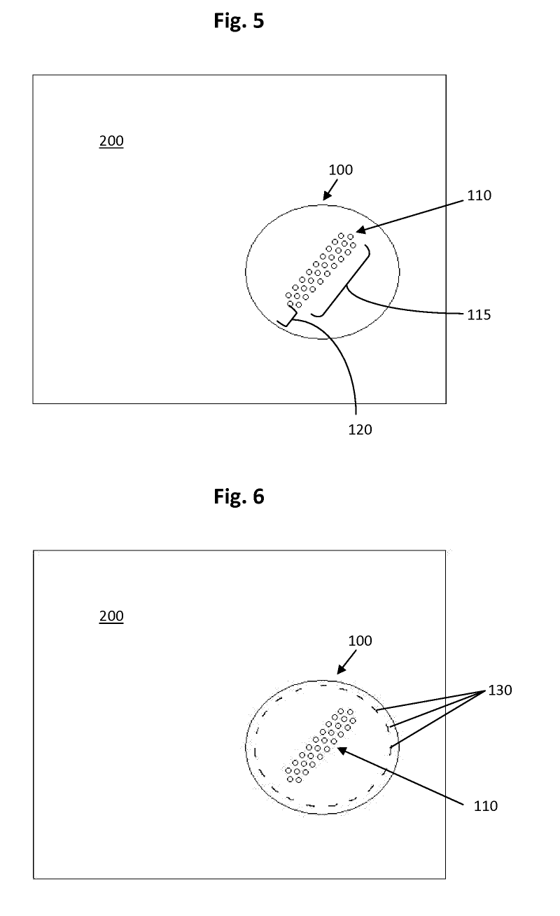

In use, a surgical mesh 100 of the above-disclosed composition may be used to both enhance and improve a wide variety of surgical procedures including but not limited to hernia repairs, repair of anatomical defects of the abdominal wall, diaphragm, and chest wall, correction of defects in the genitourinary system, and repair of traumatically damaged organs such as the spleen, liver, or kidney. FIG. 4 depicts an exemplary patient bodily surface 200 having a surgical site, repair, or incision 210 therein. As shown in FIG. 5, a surgical mesh 100 of the present invention having a plurality of apertures 110 may then be disposed above, below, or near the surgical site, repair, or incision 210. The cluster of the plurality of apertures 110 are disposed within the surgical mesh 100 such that, as shown in FIG. 3, the plurality of apertures 110 may be placed immediately adjacent or near the surgical site, repair, or incision 210. Such placement of the plurality of apertures 110 allows for reduction of foreign body inoculum at a surgical site 210, may decrease peri-incisional inflammation and incisional separation, and only decreases material density in the region near the incision 210 while allowing the surgical mesh 100 to retain its full strength and functionality. After placement of the surgical mesh 100 and as shown in FIG. 6, the surgical mesh 100 may be secured or fixed to surrounding patient tissue via any conventional means including but not limited to one or more sutures, tacks, or any other surgical fasteners 130 known within the art. In a preferred embodiment, the sutures, tacks, or other surgical fasteners 130 may be placed within the peripheral edges, circumference, or outer border 120 of the surgical mesh 100.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the full breadth of the present disclosure, and not by only the few illustrative examples given.

What is claimed is:

1. A surgical material for placement against a surgical site for reducing inoculums and other foreign bodies, said surgical material comprising:
a mesh layer for placement against said surgical site;
a plurality of apertures disposed within and extending through said mesh layer for decreasing said inoculums and other foreign bodies;
wherein said plurality of apertures both decreases the density of said mesh layer and decreases the surface area of said mesh layer exposed to said surgical site.

2. The surgical material of claim 1, wherein said plurality of apertures is formed by application of a laser energy source on said mesh layer, wherein one or more loose ends of said mesh layer disposed about said plurality of apertures are sealed by said laser energy source.

3. The surgical material of claim 1, wherein said plurality of apertures is formed by application of an ultrasound energy source on said mesh layer, wherein one or more loose ends of said mesh layer disposed about said plurality of apertures are sealed by said ultrasound energy source.

4. The surgical material of claim 1, wherein said plurality of apertures is formed by application of a hot dye on said mesh layer, wherein one or more loose ends of said mesh layer disposed about said plurality of apertures are sealed by said hot dye.

5. The surgical material of claim 1, wherein said plurality of apertures is formed by an accessory braid or weave pattern disposed within said mesh layer.

6. The surgical material of claim 1, said surgical material further comprising:
one or more secondary apertures disposed within and extending through said mesh layer for decreasing said inoculums and other foreign bodies at one or more locations not immediately adjacent said surgical site.

7. A surgical material for placement against a surgical site for reducing inoculums and other foreign bodies, said surgical material comprising:
a mesh layer for placement against said surgical site, said mesh layer comprising;
a permanent material; and
an absorbable material;
wherein said mesh layer is formed by braiding or weaving said permanent material with said absorbable material and said absorbable material is capable of allowing tissue ingrowth within said mesh layer thereby decreasing the density of said mesh layer and decreasing the surface area of said mesh layer that is exposed to said surgical site.

8. The surgical material of claim 7, wherein said permanent material comprises a more reactive material and said absorbable material comprises a less reactive material and said more reactive material creates a greater foreign body reaction than said less reactive material.

9. The surgical material of claim 7, wherein said permanent material comprises polypropylene fibers.

10. The surgical material of claim 7, wherein said absorbable material is selected from the group consisting of blends and/or copolymers of polyglycolic acid, lactic acid, and caprolactone.

11. The surgical material of claim 7, said surgical material further comprising:
one or more secondary apertures disposed within and extending through said mesh layer for decreasing said inoculums and other foreign bodies at one or more locations not immediately adjacent said surgical site.

12. A method of using a surgical material for placement against a surgical site to reduce inoculums and other foreign bodies, said method comprising the steps of:
providing a mesh layer; and
placing said mesh layer against said surgical site, wherein a plurality of apertures disposed within and extending through said mesh layer is disposed against said surgical site and is capable of allowing tissue ingrowth and reducing said inoculums and other foreign bodies about said surgical site.

13. The method of claim 12, wherein said plurality of apertures is formed by application of a laser energy source on said mesh layer, wherein one or more loose ends of said mesh layer disposed about said plurality of apertures are sealed by said laser energy source.

14. The method of claim 12, wherein said plurality of apertures is formed by application of an ultrasound energy source on said mesh layer, wherein one or more loose ends of said mesh layer disposed about said plurality of apertures are sealed by said ultrasound energy source.

15. The method of claim 12, wherein said plurality of apertures is formed by application of a hot dye on said mesh layer, wherein one or more loose ends of said mesh layer disposed about said plurality of apertures are sealed by said hot dye.

16. The method of claim 12, wherein said plurality of apertures is formed by an accessory braid or weave pattern disposed within said mesh layer.

17. The method of claim 12, wherein said mesh layer further comprises:
one or more secondary apertures disposed within and extending through said mesh layer for decreasing said inoculums and other foreign bodies at one or more locations not immediately adjacent said surgical site.

18. The method of claim 12, wherein said mesh layer further comprises:
a permanent material; and
an absorbable material;
wherein said mesh layer is formed by braiding or weaving said permanent material and said absorbable material and said absorbable material is capable of allowing for tissue ingrowth within said mesh layer thereby decreasing the density of said mesh layer and decreasing the surface area of said mesh layer that is exposed to said surgical site.

19. The method of claim 18, wherein said permanent material comprises a more reactive material and said absorbable material comprises a less reactive material and said more reactive material creates a greater foreign body reaction than said less reactive material.

20. The method of claim 18, wherein said permanent material comprises polypropylene fibers.

21. The method of claim 18, wherein said absorbable material is selected from the group consisting of blends and/or copolymers of polyglycolic acid, lactic acid, and caprolactone.

22. The method of claim 18, wherein said mesh layer further comprises:
one or more secondary apertures disposed within and extending through said mesh layer for decreasing said inoculums and other foreign bodies at one or more locations not immediately adjacent said surgical site.
